SEQUENCE LISTING (5 sequences)

SEQ ID NO: 1 - protein, 738 aa, Artificial Sequence
  Synthetic rAAVRec2
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 738 aa, Artificial Sequence
  Synthetic rAAVRec3
MAADGYLPDWLEGNLSEGIREWWDLKPGAPKPKANQQKQDDGRGLVLPGYRYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - protein, 735 aa, Adeno-associated virus
  misc_feature: AAV2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 4 - protein, 724 aa, Adeno-associated virus
  misc_feature: AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 5 - DNA, 2217 bp, Artificial Sequence
  Synthetic nucleotide sequence of rAAVRec3 and      rAAVRec2
atggcggcggatggctatctgccggattggctggaaggcaacctgagcgaaggcattcgcgaatggtgggatctgaaaccgggcgcgccgaaaccgaaagcgaaccagcagaaacaggatgatggccgcggcctggtgctgccgggctatcgctatctgggcccgtttaacggcctggataaaggcgaaccggtgaacgaagcggatgcggcggcgctggaacatgataaagcgtatgatcagcagctgaaagcgggcgataacccgtatctgcgctataaccatgcggatgcggaatttcaggaacgcctgcaggaagataccagctttggcggcaacctgggccgcgcggtgtttcaggcgaaaaaacgcgtgctggaaccgctgggcctggtggaagaagcggcgaaaaccgcgccgggcaaaaaacgcccggtggaaccgagcccgcagcgcagcccggatagcagcaccggcattggcaaaaaaggccagcagccggcgaaaaaacgcctgaactttggccagaccggcgatagcgaaagcgtgccggatccgcagccgattggcgaaccgccggcgggcccgagcggcctgggcagcggcaccatggcggcgggcggcggcgcgccgatggcggataacaacgaaggcgcggatggcgtgggcagcagcagcggcaactggcattgcgatagcacctggctgggcgatcgcgtgattaccaccagcacccgcacctgggcgctgccgacctataacaaccatctgtataaacagattagcaacggcaccagcggcggcagcaccaacgataacacctattttggctatagcaccccgtggggctattttgattttaaccgctttcattgccattttagcccgcgcgattggcagcgcctgattaacaacaactggggctttcgcccgaaacgcctgagctttaaactgtttaacattcaggtgaaagaagtgacccagaacgaaggcaccaaaaccattgcgaacaacctgaccagcaccattcaggtgtttaccgatagcgaatatcagctgccgtatgtgctgggcagcgcgcatcagggctgcctgccgccgtttccggcggatgtgtttatgattccgcagtatggctatctgaccctgaacaacggcagccaggcggtgggccgcagcagcttttattgcctggaatattttccgagccagatgctgcgcaccggcaacaactttgaatttagctatacctttgaagatgtgccgtttcatagcagctatgcgcatagccagagcctggatcgcctgatgaacccgctgattgatcagtatctgtattatctgagccgcacccagagcaccggcggcacccagggcacccagcagctgctgtttagccaggcgggcccggcgaacatgagcgcgcaggcgaaaaactggctgccgggcccgtgctatcgccagcagcgcgtgagcaccaccctgagccagaacaacaacagcaactttgcgtggaccggcgcgaccaaatatcatctgaacggccgcgatagcctggtgaacccgggcgtggcgatggcgacccataaagatgatgaagaacgcttttttccgagcagcggcgtgctgatgtttggcaaacagggcgcgggccgcgataacgtggattatagcagcgtgatgctgaccagcgaagaagaaattaaaaccaccaacccggtggcgaccgaacagtatggcgtggtggcggataacctgcagcagaccaacaccggcccgattgtgggcaacgtgaacagccagggcgcgctgccgggcatggtgtggcagaaccgcgatgtgtatctgcagggcccgatttgggcgaaaattccgcataccgatggcaactttcatccgagcccgctgatgggcggctttggcctgaaacatccgccgccgcagattctgattaaaaacaccccggtgccggcggatccgccgaccacctttaaccagagcaaactgaacagctttattacccagtatagcaccggccaggtgagcgtggaaattgaatgggaactgcagaaagaaaacagcaaacgctggaacccggaaattcagtataccagcaactattataaaagcaccagcgtggattttgcggtgaacaccgaaggcgtgtatagcgaaccgcgcccgattggcacccgctatctgacccgcaacctgtaa

(19) United States
(12) Patent Application Publication
(10) Pub. No.: US 2019/0085358 A1
(43) Pub. Date: Mar. 21, 2019

(54) RECOMBINANT ADENO-ASSOCIATED VECTORS

(71) Applicant: Ovid Therapeutics Inc., New York, NY (US)

(72) Inventor: Matthew DURING, Weston, CT (US)

(21) Appl. No.: 16/108,393

(22) Filed: Aug. 22, 2018

Related U.S. Application Data
(60) Provisional application No. 62/550,458, filed on Aug. 25, 2017.

(51) Int. Cl. C12N 15/86; A61K 48/00

(52) U.S. Cl. CPC C12N 15/86; C12N 2750/14143; A61K 48/005

(57) ABSTRACT

Adeno-associated virus (AAV) vectors and uses thereof are provided. More specifically, AAV vectors are provided that show specific tropism for certain target tissue, such as central nervous system (CNS) and adipose tissue, and which may be used to transduce cells for introduction of genes of interest into the target tissues. Pharmaceutical compositions are also provided that include AAV vectors and a pharmaceutically acceptable excipient, diluent or carrier.

### 1. TECHNICAL FIELD

The present disclosure relates generally to adeno-associated virus (AAV) vectors and uses thereof. More specifically, the present disclosure relates to vectors that show specific tropism for certain target tissue, such as central nervous system (CNS) and adipose tissue, and may be used to transduce cells for introduction of genes of interest into the target tissues.

### 2. BACKGROUND

Adeno-associated virus (AAV) is a single-stranded DNA virus that is currently being utilized for gene therapy applications. AAV is a member of the family Parvoviridae, genus Dependovirus. The AAV genome, which is approximately 4.7 kb long (1, 2), contains two open reading frames (ORF), rep and cap, flanked by inverted terminal repeat elements (ITR) (3). There are 11 known serotypes of AAV with different cellular targets and antigenic properties (Wu et al., 2006). Recently, about 100 genomic variants of these primary AAV serotypes have been discovered (6).

The first AAV vectors were generated 30 years ago based on AAV2 (Tratschin et al., 1984; Hermonat et al., 1984). Vectors based on AAV2 (AAV2) have been the most studied and are currently used in clinical trials for numerous diseases including cystic fibrosis, hemophilia B, prostate and melanoma cancers, Canavan disease, Alzheimer's, Parkinson's, muscular dystrophy, rheumatoid arthritis, and HIV vaccines (15). These vectors have been shown in animal models to deliver genes to broad range of cells in muscle, brain, retina, liver, and lung (5, 16-22). Problems associated with current AAV vector systems include unintended transduction of certain tissues, and lack of efficient transduction of the tissue of interest. Accordingly, safe and efficient gene delivery to specific tissues of interest, such as CNS tissue, remains a significant challenge in the field.

### 3. SUMMARY

Recombinant AAV vector serotypes in accordance with the present disclosure, referred to as rAAVRec2 and rAAVRec3, are provided. The rAAVRec2 and rAAVRec3 vectors are found to have an increased tropism to adipose and CNS tissue, respectively. The present AAV vectors contain modifications of amino acid residues in the capsid VP1, VP2 and VP3 regions as compared to those found in wild type AAV2 and AAV5 (FIG. 1A). Additionally, the rAAVRec3 is able to be propagated to high virus titre levels. Such growth properties are advantageous for efficient and less costly generation of useful viral stocks.

In embodiments, novel rAAV capsid proteins, as well as nucleic acid molecules coding for the novel capsids are provided. In a specific embodiment, novel capsid amino acid sequences include those of FIG. 1A (rAAVRec2 and rAAVRec3). In aspects of this disclosure, nucleic acid molecules encoding the presently disclosed virus capsids and capsid proteins are provided. Nucleic acid molecules encoding the present capsid proteins include those of FIG. 1B (rAAVRec3). Further provided are vectors including nucleic acid molecules encoding the rAAVRec2 and rAAVRec3 capsid proteins, and cells (in vivo or in vitro) containing the presently disclosed rAAVRec2 and rAAVRec3 nucleic acids and/or vectors. Such nucleic acids, vectors, and cells can be used, for example, for directed expression of rAAVRec2 and rAAVRec3 capsid proteins. Such protein expression may be used to develop reagents (e.g., helper constructs or packaging cells) for the production of the novel AAV vectors as described herein. Further provided are recombinant viruses (virions) wherein the capsid protein of said viruses are the capsid protein of rAAVRec2 or the capsid protein of rAAVRec3. Such viruses may be used to transduce a heterologous nucleic acid of interest into a target cell or tissue.

In aspects of the present disclosure, a method for delivering or transferring a heterologous polynucleotide sequence into a mammal or a cell of a mammal is provided, including the step of administering an adeno-associated virus (AAV) vector in accordance with the present disclosure, the vector including one or more of the rAAVRec2 and rAAVRec3 VP1, VP2, or VP3 capsid proteins set forth in FIG. 1A and a heterologous polynucleotide sequence, to said mammal or a cell of said mammal, thereby delivering or transferring the heterologous polynucleotide sequence into the mammal or cell of the mammal. In embodiments, the AAV vector is rAAVRec2 and the mammalian cell or cell of the mammal is a cell of adipose tissue, for example an adipocyte cell. In embodiments, the AAV is rAAVRec3 and the mammalian cell or cell of the mammal is a cell of the CNS, for example a neuronal cell.

In a further aspect of the present disclosure, a method of treating a mammal deficient in protein expression or function is provided, including the step of: administering an adeno-associated virus (rAAV) vector, encoding one or more of the capsid proteins of rAAVRec3, the vector also including a heterologous polynucleotide sequence encoding a polypeptide that can correct for the deficient protein expression or function, in an amount wherein the polypeptide is expressed in the mammal. In embodiments, the rAAV is rAAVRec3 and the mammalian cell or cell of the mammal is a cell of the CNS, for example a neuronal cell. For gene therapy involving cells of the CNS, the heterologous polynucleotide sequence may encode, for example, a wild type hamartin (TSC1) or tuberin (TSC2) protein for treatment of tuberous sclerosis complex. In another embodiment, the heterologous polynucleotide sequence may encode the wild the SMA (SMA) protein for treatment of spinal muscular atrophy.

In embodiments, a method of treating a mammal deficient in protein expression or function is provided, including the step of: administering adeno-associated virus (AAV) vector including the capsid of rAAVRec2, the vector including a heterologous polynucleotide encoding a polypeptide that can correct for the deficient protein expression or function, in an amount of wherein the polypeptide is expressed in the mammal. In embodiments, the rAAV is rAAVRec2 and the mammalian cell or cell of the mammal is a cell of adipose tissue, for example an adipocyte.

The loss of body fat in inherited lipodystrophies can be caused by defects in the development and/or differentiation of adipose tissue as a consequence of mutations in a number of genes. In embodiments of the present disclosure, the heterologous polynucleotide sequence may encode wild-type counterparts for the defective genes associated with lipodystorphies. Accordingly, for gene therapy involving cells of adipose tissue, the heterologous polynucleotide sequence may encode, for example, a wild-type PPARG, AGPAT2, AKT2, BSCL2, lamin A/C, nuclear lamina proteins and ZMPSTE24 genes.

In a further aspect of the present disclosure, pharmaceutical compositions are provided that include AAVRec3 and rAAVRec2 vectors and a pharmaceutically acceptable excipient, diluent or carrier. In another aspect of the present disclosure, kits including one or more of the rAAVRec3 and rAAVRec2 vector compositions are provided together with one or more pharmaceutically-acceptable excipients, carriers, diluents, adjuvants, and/or other components, and instructions for using the rAAV vectors in the treatment of disorders in a subject, and may typically further include containers prepared for convenient commercial packaging.

### 4. BRIEF DESCRIPTION OF THE DRAWINGS

Various embodiment of the present recombinant vectors, proteins compositions and methods are described herein with reference to the drawings wherein:

FIG. 1A shows VP protein alignment of rAAVRec3, rAAVRec2, AAV2 and AAV5. Amino acid sequence of rAAVRec 3 and rAAVRec2;

FIG. 1B shows the nucleotide sequence of rAAVRec3 and rAAVRec2;

FIG. 2A-B. FIG. 2A shows GFP expression driven by a CAG promoter packaged into AAV1, AAV8, AAV9 and rAAVRec1-4. 2.5× magnification views of mice brain. Shown are sections (a) GFP expression at injection site in striatum (column 2). FIG. 2B. The total volume of transduced area within the brain.

FIG. 3 shows transduction of neuronal or glial cell populations by rAAV vectors. Merged GFP fluorescence (column 1, in green). NeuN or GFAP (column 2, in red) show that some GFP-positive cells were also stained for NeuN or GFAP, resulting in yellow merged fluorescence (column 3).

### 5. DETAILED DESCRIPTION

### 5.1 Recombinant AAV Serotypes

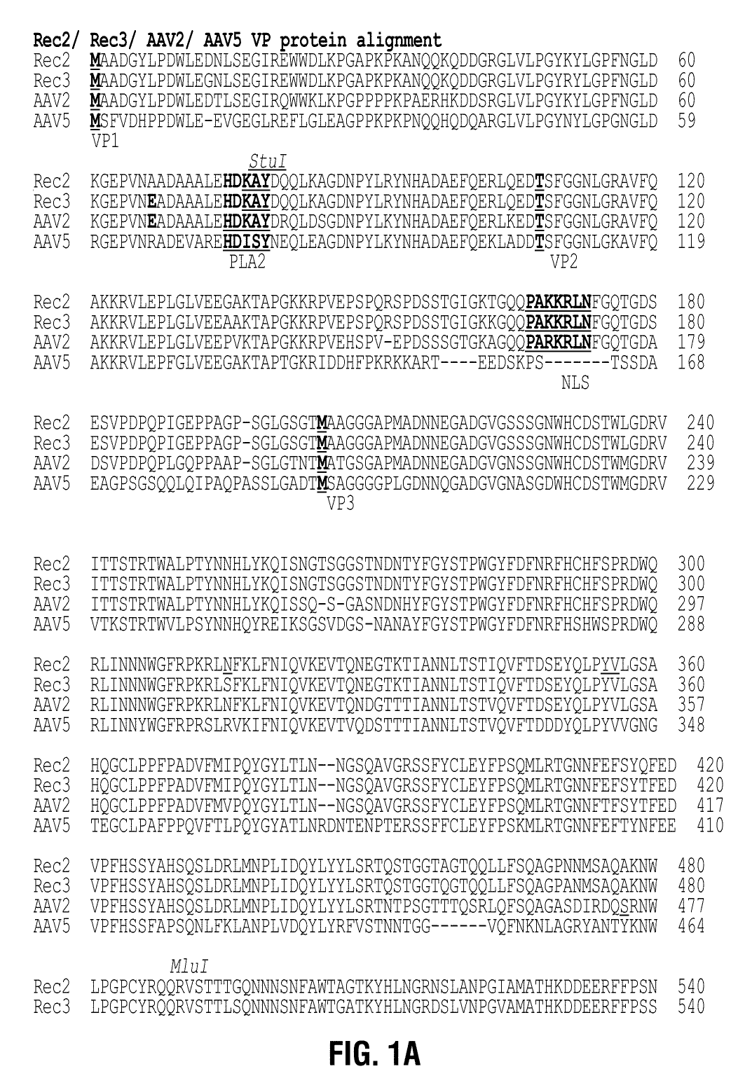

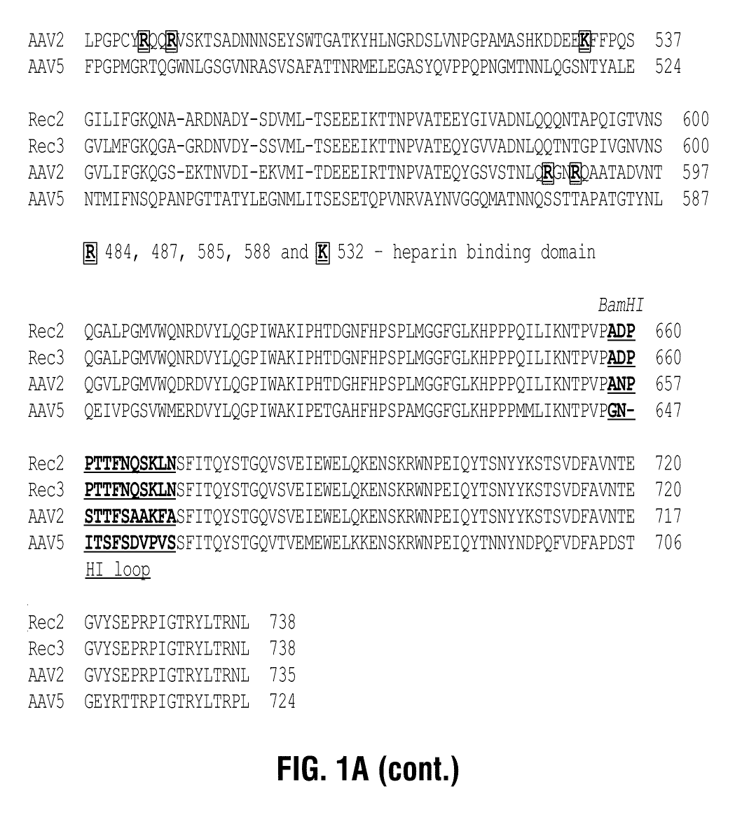

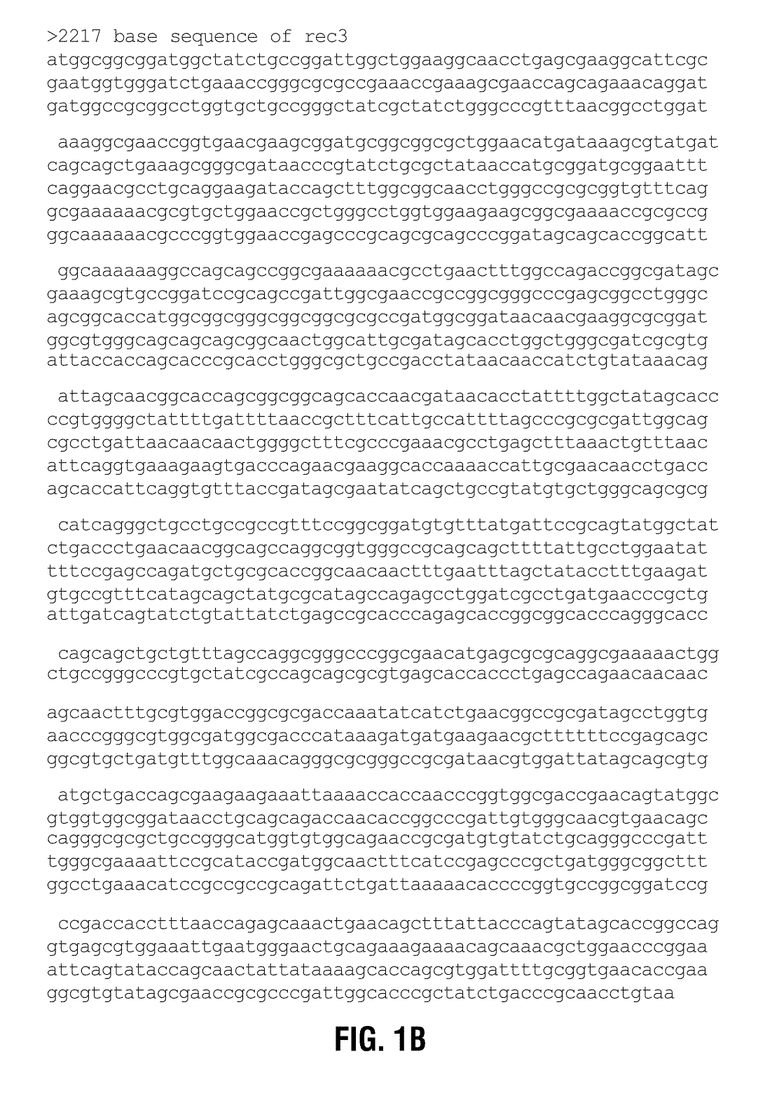

Recombinant AAV vector serotypes, referred to as rAAVRec2 and rAAVRec3, are provided. In embodiments, the present AAV serotypes include one or more of the hybrid VP1, VP2 and VP3 amino acid sequences presented in FIG. 1A. The present rAAV vectors contain modifications of amino acid residues in the capsid encoding VP1, VP2 and VP3 regions as compared to wild type AAV2 and AAV5 (FIG. 1A). The disclosed recombinant serotypes display an improved efficiency in transduction of a variety of cells, tissues and organs of interest. Specifically, the rAAVRec2 serotype demonstrates a higher efficiency in transduction of cells of adipose tissue while the rAAVRec3 serotype demonstrates a higher efficiency in transduction of cells of the central nervous system (CNS). Additionally, the rAAVRec3 virus is able to be propagated to high titres as compared to other AAV viruses (See, Table 1).

The rAAV capsid proteins disclosed herein, are capable of preferentially transducing cells of the CNS (rAAVRec3) or adipose tissue (rAAVRec2). In embodiments, the rAAV capsid proteins include the VP1-3 amino acid sequences of rAAVRec2 and AAVRec3 as presented in FIG. 1. In some embodiments, modified rAAVRec2 and rAAVRec3 capsid proteins are provided having amino acid sequences that are at least 91%, 92%, 93%, 94%, 95%, 96%, 97%, 98% or 99% identical to the amino acid sequence of rAAVRec 2 and rAAVRec3 capsid proteins (FIG. 1A). Such modified capsid proteins substantially retain the tropism observed for rAAVRec2 and rAAVRec3. For example, a virus particle including the modified capsid or modified capsid protein can substantially retain the CNS tropism profile of a rAAVRec3 virus particle including a rAAVRec3 capsid or capsid protein of FIG. 1A. Further, a virus particle including the modified capsid or modified capsid protein can substantially retain the adipose tissue tropism profile of a rAAVRec2 virus particle including a rAAVRec2 capsid or capsid protein of FIG. 1A.

Nucleic acid molecules encoding one or more of the AAV capsid proteins (VP1-3) of FIG. 1 are provided. In embodiments, the nucleic acid molecule includes that of FIG. 1B. In embodiments, the AAV capsid encoding sequence is at least 91%, 92%, 93%, 94%, 95%, 96%, 97%, 98% or 99% identical to the nucleotide sequence of FIG. 1B and encodes for AAV capsid proteins with a tropism for cells of the CNS.

As is known in the art, a number of different programs can be used to identify whether a nucleic acid or polypeptide has sequence identity to a known sequence. Percent identity as used herein means that a nucleic acid or fragment thereof shares a specified percent identity to another nucleic acid, when optimally aligned (with appropriate nucleotide insertions or deletions) with the other nucleic acid (or its complementary strand), using BLASTN. To determine percent identity between two different nucleic acids, the percent identity is to be determined using the BLASTN program “BLAST 2 sequences”. This program is available for public use from the National Center for Biotechnology Information (NCBI). Percent identity or similarity when referring to polypeptides, indicates that the polypeptide in question exhibits a specified percent identity or similarity when compared with another protein or a portion thereof over the common lengths as determined using BLASTP. This program is also available for public use from the National Center for Biotechnology Information (NCBI).

The presently disclosed AAV capsid proteins include full-length rAAVRec2 and rAAVRec3 VP-1, VP-2 and VP-3 sequences, as well as functional protein fragments, modified forms or sequence variants so long as the fragment, modified form or variant retains the function and tissue tropism of the full-length protein. Additionally, the AAV capsid proteins of FIG. 1A can be further modified to incorporate modifications known in the art to impart desired properties. In embodiments, the capsid protein(s) can be modified to incorporate sequences (“tags”) that facilitate purification and/or detection. Such tags include for example, polyhistidine (HIS) or glutathione S-transferase (GST), Glu-Glu, and streptavidin binding protein tags. Methods of inserting such modifications into the AAV capsid are known in the art.

The present disclosure further relates to expression vectors including nucleic acid molecules encoding the rAAVRec2 and rAAVRec3 capsid proteins. Nucleic acid molecules encoding the rAAVRec2 and rAAVRec3 capsid proteins may be used as part of an expression vector, which may be isolated and purified. Such expression vectors may be isolated and purified for use as helper vectors for generation of rAAV stocks. Such viral stocks may contain a vector genome having a heterologous nucleic acid of interest. The sequences may also be used to transduce cells for production of rAAVRec2 and rAAVRec3 capsid proteins. Nucleic acid molecules coding for rAAVRec2 and rAAVRec3 capsid proteins can be inserted separately or together into an expression vector using any of the methods described below for their expression. The sequences may also be truncated such as partial VP1-VP2-VP3 or VP1-VP3 or VP1-VP1-VP2-VP3.

In embodiments, vectors for expression of the rAAVRec2 and rAAVRec3 proteins include, but are not limited to a plasmid, phage, viral vector (e.g., AAV vector, an adenovirus vector, a herpesvirus vector, or a baculovirus vector), mammalian vector, bacterial artificial chromosome (BAC), or yeast artificial chromosome (YAC). The vector can include an AAV vector including a 5′ and/or 3′ terminal repeat (e.g., 5′ and/or 3′ AAV terminal repeat). The presently disclosed vectors may further include expression control elements, such as transcription/translation control signals, origins of replication, polyadenylation signals, internal ribosome entry sites (IRES), promoters, enhancers, and the like.

The AAV vectors described herein may be used for transducing specific types of mammalian cells, for example, cells of the CNS and adipose tissue for introduction of genes of interest into target tissues. Cells of the CNS include, for example, neurons and glia cells. Cells of adipose tissue include adipocytes. Accordingly, the present disclosure contemplates AAV-based expression systems, and vectors wherein the AAV expression vectors include at least a first heterologous nucleic acid molecule that encodes a therapeutic peptide, protein, polypeptide, or an antisense molecule.

Genetic disease is associated with the presence of defective genes that either fail to produce a protein product, produce a protein product that fails to function properly, or produce a dysfunctional protein product that interferes with the proper function of the cell. Gene transfer can be used in providing therapy for such genetic disease. Accordingly, in aspects of the present disclosure, the present rAAV vectors include a heterologous nucleic acid that may encode a therapeutically functional protein or a polynucleotide that inhibits production or activity of a dysfunctional protein.

The ability to target rAAV expression vectors to neurons may be particularly useful to treat diseases or disorders involving neuron dysfunction including for example genetic diseases of the CNS. In embodiments, the present rAAVRec3 vectors include a heterologous nucleic acid for introduction into cells of the brain such as, for example, neuronal cells. In embodiments, the vectors are useful to express a polypeptide or nucleic acid that provides a beneficial effect to neurons, e.g., to promote growth and/or differentiation of neurons.

In embodiments, the present rAAV vectors may be engineered to treat tuberous sclerosis complex (TSC) patients. Tuberous sclerosis complex is a genetic disorder that can affect the brain, causing seizures, behavioral problems such as hyperactivity and aggression, and intellectual disability or learning problems. Some TSC afflicted children have features of autism. Additionally, benign brain tumors can also develop in people with TSC.

TSC is an autosomal dominant genetic disease caused by mutations in TSC1 or TSC2 genes which encode the hamartin and tuberin proteins, respectively. Accordingly, the presently described rAAVRec3 vectors may be engineered and used in gene therapy applications to transduce the wild-type hamartin or tuberin genes into neuronal cells of TSC patients. In embodiments, an AAV vector is provided including a heterologous nucleic acid that codes for the wild-type hamartin protein. In other embodiments, an AAV vector is provided including a heterologous nucleic acid that codes for the wild-type tuberin protein. (See, Kwiatkowski et al., 2010. Tuberous Sclerosis Complex: Genes, Clinical Features and Therapeutics. Wiley-Blackwell, Weinheim, Germany).

In embodiments, the presently described rAVVRec3 vectors may be used to treat spinal muscular atrophy (SMA) Type 1. SMA is a genetic disease affecting the part of the nervous system that controls voluntary muscle movement. SMA involves the loss of nerve cells called motor neurons in the spinal cord. The genetic disorder is caused by a deficiency of the motor neuron protein called SMN1. Accordingly, the presently disclosed rAAVRec3 vectors may be engineered and used in gene therapy applications to transduce the wild-type SMN1 gene into neuronal cells of SMN patients. In embodiments, a rAAVRec3 vector is provided including a heterologous nucleic acid that codes for the wild-type SMN1 protein. (See, Lefebvre S, et al. Cell. 1995; 80:155-165: Wetz and Sahin Ann NY Acad Sci 2016 1366 (1):5-19).

The ability to target AAV expression vectors to adipose tissue may also be useful to treat diseases or disorders involving adipocyte dysfunction including, for example, genetic diseases such as lipodystrophies. Inherited lipodystrophies can be caused by defects in the development and/or differentiation of adipose tissue as a consequence of mutations in a number of genes. Such genes include, but are not limited to, defective PPARG, AGPAT2, AKT2, BSCL2, lamin A/C, nuclear lamina proteins and ZMPSTE24 genes. In embodiments, the presently described rAAV vectors contain a heterologous nucleic acid for introduction into cells of adipose such as, for example, adipocytes. In embodiments, the vectors are useful to express a polypeptide or nucleic acid that provides a beneficial effect to adipocytes, e.g., to promote growth and/or differentiation of adipocytes. In an embodiment, the heterologous polynucleotide sequence may encode a wild-type counterpart for the defective genes associated with lipodystorphies. Accordingly, for gene therapy involving cells of adipose tissue, the heterologous polynucleotide sequence may encode, for example, a wild-type PPARG, AGPAT2, AKT2, BSCL2, lamin A/C, nuclear lamina proteins and ZMPSTE24 genes.

It will be understood by those skilled in the art that the heterologous nucleic acid(s) of interest may be operably associated with appropriate control sequences. For example, the heterologous nucleic acids may be operably associated with expression control elements, such as transcription/translation control signals, origins of replication, polyadenylation signals, internal ribosome entry sites (IRES), promoters, enhancers, and the like. Such elements also optionally include a transcription termination signal. A particular non-limiting example of a transcription termination signal is the SV40 transcription termination signal. Additionally, the heterologous nucleic acid molecule may include AAV 5′ and/or 3′ terminal repeats (e.g., 5′ and/or 3′ AAV terminal repeat) for encapsidation of the molecule into the novel AAV capsids. In embodiments wherein the heterologous nucleic acid is transcribed and then translated in the target cells, specific initiation signals are generally employed for efficient translation of inserted protein coding sequences. These exogenous translational control sequences, which may include the ATG initiation codon and adjacent sequences, can be of a variety of origins, both natural and synthetic.

A variety of promoter/enhancer elements may be used depending on the level and tissue-specific expression desired. The promoter/enhancer may be constitutive or inducible, depending on the pattern of expression desired. The promoter/enhancer element is generally chosen so that it will function in the target cell(s) of interest. In representative embodiments, the promoter/enhancer element is a mammalian promoter/enhancer element. In a specific embodiment the promoter/enhancer is an element that functions specifically in cells of the CNS or cells of adipose tissue. The promoter/enhance element may also be constitutive or inducible.

The present disclosure provides rAAVRec2 and rAAVRec3 virus particles (i.e., virions) wherein the virus particle packages a vector genome, optionally an AAV vector genome that contains a heterologous nucleic acid of interest. Such virus particles show a tropism for adipose tissue (rAAVRec2) or CNS tissue (rAAVRec3). Methods for propagation of virus particles are well known to persons skilled in the art (See, for example, Shin et al., Methods Mol. Biol. 798; 267-284). AAV can be propagated both as lytic virus and as a provirus. For lytic growth, AAV requires co-infection with a helper virus such as, for example, adenovirus or herpes simplex viruses. In the absence of helper virus, AAV will exist as an integrated provirus. When cells carrying an AAV provirus are subsequently infected with a helper, the integrated AAV genome is rescued and a productive lytic cycle occurs. Alternatively, the helper virus functions may be provided by a packaging cell with the helper genes integrated in the chromosome or maintained as a stable extrachromosomal element.

For propagation of virus particles, the cell is typically a cell that is permissive for AAV viral replication. Any suitable cell known in the art may be employed, such as mammalian cells. Also suitable are trans-complementing packaging cell lines that provide functions deleted from a replication-defective helper virus, e.g., 293 cells or other E1A trans-complementing cells.

In embodiments, the methods of producing recombinant virus particles includes providing to a cell in vitro, (a) a vector genome including (i) a heterologous nucleic acid, and (ii) packaging signal sequences sufficient for the encapsidation of the vector genome into virus particles (such as AAV terminal repeats), and (b) AAV rep and AAV cap sequences sufficient for replication and encapsidation of the vector genome into viral particles. The vector genome nucleic acid and AAV rep and cap sequences are provided under conditions such that recombinant virus particles including the vector genome are packaged within the capsid produced in the cell.

In some embodiments, the viral particles are isolated and purified, such as, for example, for in vivo administration to increase efficacy and reduce contamination. The present packaging methods may be employed to produce high titer stocks of virus particles. In embodiments, the virus stock may have a titer of at least about 10⁵ transducing units (tu)/ml, at least about 10⁶ tu/ml, at least about 10⁷ tu/ml, at least about 10⁸ tu/ml, at least about 10⁹ tu/ml, or at least about 10¹⁰ tu/ml.

### 5.2. Uses of the Recombinant Viral Vector

The present disclosure provides rAAVRec2 and rAAVRec3 vectors and viruses (virions) that show a specific tropism for certain target tissue, such as CNS and adipose tissue. In embodiments, rAAV vectors and virions are used for transduction of mammalian host cells including, for example, mammalian cells of the CNS and cells of adipose tissue. The rAAVRec2 and rAAVRec3 vectors or viruses can be used to introduce or deliver heterologous nucleic acids stably or transiently into cells and progeny thereof. Heterologous nucleic acids include any polynucleotide, such as a gene that encodes a polypeptide or protein or a polynucleotide that is transcribed into an inhibitory polynucleotide.

The rAAVRec2 and rAAVRec3 vectors disclosed herein are useful in methods for delivering a nucleotide sequence to a subject in need thereof, for example, to express a therapeutic polypeptide or nucleic acid in vivo in the subject. The subject may be in need of the polypeptide or nucleic acid because the subject has a deficiency of the polypeptide, or because the production of the polypeptide or nucleic acid in the subject may impart some therapeutic effect.

Disclosed herein are methods for delivering a heterologous polynucleotide sequence into a mammal or a cell of a mammal. In embodiments, the method includes administering a rAAV vector that includes a heterologous nucleic acid to a mammal or a cell of a mammal under suitable conditions to deliver the heterologous polynucleotide sequence into the mammal or the cell of a mammal, thereby delivering the heterologous polynucleotide. In one aspect, the method allows delivery of the heterologous nucleic acid into the mammal and/or cell. In another aspect, the method allows delivery of the heterologous polynucleotide into the mammal and/or cell, and subsequent transcription of the heterologous polynucleotide thereby forming a transcript. In a further aspect, the method allows delivery of the heterologous polynucleotide into the cell, subsequent transcription to form a transcript and subsequent translation to form a gene product (protein).

In one aspect, a method of delivering a nucleic acid of interest to cells of adipose tissue is provided, the method including the step of contacting the cells of adipose tissue with the rAAVRec2 particle disclosed herein. In another aspect, a method is provided of delivering a nucleic acid of interest to adipose tissue in a mammalian subject, the method including the step of administering an effective amount of the rAAVRec2 virus particle or pharmaceutical formulation in accordance with the present disclosure to a mammalian subject.

In another aspect, a method of delivering a nucleic acid of interest to a cell of the CNS is provided, the method including the step of contacting the neuron with a rAAVRec3 particle in accordance with the present disclosure. In another aspect, a method of delivering a nucleic acid of interest to brain tissue in a mammalian subject is provided, the method including the step of administering an effective amount of the rAAVRec3 virus particle or pharmaceutical formulation to a mammalian subject.

In one embodiment, the method includes the step of administering an amount of the present rAAV vector to a mammalian subject, said vector including a heterologous nucleic acid encoding a protein wherein the heterologous nucleic acid is operably linked to an expression control element conferring transcription of said nucleic acid, wherein said protein is expressed in the mammal. In particular aspects, expression of the protein provides a therapeutic benefit to the mammal.

The tropism of a rAAVRec3 vector for central nervous system tissue may be exploited for the treatment of brain disorders. The rAAVRec3 vector may be employed to deliver a nucleotide sequence of interest to cells of the CNS to produce a polypeptide or nucleic acid in vitro or for ex vivo gene therapy. In an embodiment, the vectors are useful to express a polypeptide or nucleic acid that provides a beneficial effect to cells of the CNS, e.g., to promote growth and/or differentiation of neurons. The ability to target vectors to neurons may be useful to treat diseases or disorders involving neurons dysfunction.

In an embodiment, a method of treating a neurological disease or disorder in a subject includes the step of administering a rAAVRec3 vector capable of selectively transducing cells of the CNS. There are many neurological diseases or disorders that are well known to one of skill in the art such as a disease or disorder of the brain, spinal cord, ganglia, motor nerve, sensory nerve, autonomic nerve, optic nerve, retinal nerve, and auditory nerve. Brain diseases or disorders may include cancer or other brain tumor, inflammation, bacterial infections, viral infections, including rabies, amoeba or parasite infections, stroke, paralysis, neurodegenerative disorders such as Alzheimer's Disease, Parkinson's Disease, or other dementia or reduction in cognitive functioning, plaques, encephalopathy, Huntington's Disease, aneurysm, genetic or acquired malformations, acquired brain injury, Tourette Syndrome, narcolepsy, muscular dystrophy, tremors, cerebral palsy, autism, Down Syndrome, attention deficit and attention deficit hyperactivity disorder, chronic inflammation, epilepsy, coma, meningitis, multiple sclerosis, myasthenia gravis, various neuropathies, restless leg syndrome, and Tay-Sachs disease.

In one aspect, the compositions disclosed herein may be used to treat tuberous sclerosis complex (TSC) patients. TSC is an autosomal dominant genetic disease caused by mutations in TSC1 or TSC2 genes which encode hamartin and tuberin, respectively. The rAAV vectors disclosed herein may be used in gene therapy applications to transduce the wild-type hamartin or tuberin gene into the cells of TSC patients.

In another aspect, the rAVV vectors disclosed herein may be used to treat spinal muscular atrophy (SMA) Type 1 by administering a rAAVRec3 virus engineered to express the SMA transgene to a patient. SMA is a genetic disease affecting the part of the nervous system that controls voluntary muscle movement. SMA involves the loss of nerve cells called motor neurons in the spinal cord and is classified as a motor neuron disease. The genetic disorder is caused by a deficiency of the motor neuron protein called SMN.

The tropism of the rAAVRec2 vector for adipose tissue may be exploited for the treatment of adipose tissue disorders. The rAAVRec2 vector may be employed to deliver a nucleotide sequence of interest to cells of adipose tissue to produce a polypeptide or nucleic acid in vitro or for ex vivo gene therapy. The vectors are useful to express a polypeptide or nucleic acid that provides a beneficial effect to cells of the adipose tissue, e.g., to promote growth and/or differentiation of adipocytes. The ability to target vectors to adipocytes can be useful to treat diseases or disorders involving adipocyte dysfunction. For example, inherited lipodystrophies can be caused by defects in the development and/or differentiation of adipose tissue as a consequence of mutations in a number of genes including, for example, PPARG, AGPAT2, AKT2, BSCL2, lamin A/C, nuclear lamina proteins and ZMPSTE24 genes. In embodiments, the heterologous polynucleotide sequence will encode a wild-type counterparts of the defective genes associated with lipodystrophies.

In an embodiment, pharmaceutical compositions containing rAAVRec2 or rAAVRec3 vectors are provided. The present pharmaceutical compositions may contain a pharmaceutically acceptable excipient, diluent or carrier. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions including such carriers are formulated by well known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

Such compositions can be formulated by conventional methods and can be administered to the subject at a suitable dose. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of infection or disease, general health and other drugs being administered concurrently. One skilled in the art can readily determine a rAAVRec2 or rAAVRec3 vector dose range to effectively treat a patient having a particular disease or disorder based on the aforementioned factors, as well as other factors.

“Effective” amount for treatment is typically effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

Subjects appropriate for treatment include those having or at risk of producing an insufficient amount or having a deficiency in a functional gene product (protein), or produce an aberrant, partially functional or non-functional protein, which can lead to disease. Subjects appropriate for treatment also include those having or at risk of producing an aberrant, or defective protein that leads to a disease such that reducing amounts, expression or function of the aberrant, or defective protein would lead to treatment of the disease, or reduce one or more symptoms or ameliorate the disease. Target subjects therefore include subjects that have such defects regardless of the disease type, timing or degree of onset, progression, severity, frequency, or type or duration of the symptoms.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or brain). Administration can also be to a tumor (e.g., in or a near a tumor or a lymph node). The most suitable route in any given case will depend on the nature and severity of the condition being treated and on the nature of the particular vector that is being used.

In some embodiments, the rAAVRec3 vectors disclosed herein are administered directly to the CNS, e.g., the brain or the spinal cord. Any method known in the art to administer vectors directly to the CNS can be used. The rAAV vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and amygdala), limbic system, neocortex, corpus striatum, cerebrum, and inferior colliculus. The rAAV vector may be delivered into the cerebrospinal fluid by, for example, lumbar puncture. In an addition, when administration is performed intravenously, ultrasound may be applied to a target location in the patient's brain to enhance permeability of the patient's blood brain barrier at the target location for uptake of the rAAV vectors. The application of ultrasound for enhancing the permeability of the patient's blood brain barrier is disclosed in Ser. No. 62/471,635, the content of which is incorporated herein in its entirety.

In one aspect, kits including one or more of the genetically-modified rAAV vector compositions described herein together with one or more pharmaceutically-acceptable excipients, carriers, diluents, adjuvants, and/or other components, as may be employed in the formulation of particular rAAV delivery formulations, and in the preparation of therapeutic agents for administration to a subject, and in particularly, to a human. In particular, such kits may include one or more of the disclosed rAAV compositions in combination with instructions for using the viral vector in the treatment of such disorders in a subject, and may typically further include containers prepared for convenient commercial packaging. The container means for such kits may typically include at least one vial, test tube, flask, bottle, syringe or other container means, into which the disclosed rAAV composition(s) may be placed, and preferably suitably aliquoted. Where a second therapeutic polypeptide composition is also provided, the kit may also contain a second distinct container means into which this second composition may be placed. Alternatively, the plurality of therapeutic biologically active compositions may be prepared in a single pharmaceutical composition, and may be packaged in a single container means, such as a vial, flask, syringe, bottle, or other suitable single container means. The kits disclosed herein will also typically include a means for containing the vial(s) in close confinement for commercial sale, such as, e.g., injection or blow-molded plastic containers into which the desired vial(s) are retained.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

### 6. EXAMPLES

The examples provided herein are included solely for augmenting the disclosure herein and should not be considered to be limiting in any respect.

### Example I

The transgene expression of rAAVRec1-4 was compared to other natural serotypes (AAV1, AAV8, AAV9) following intrastriatal injection. An expression cassette containing the CAG promoter driving the green fluorescent protein (GFP) gene was used in all the vectors. Transgene expression was evaluated by unbiased stereological analysis of the GFP fluorescence. Among the vectors studied, rAAVRec3 vectors produced the highest level of expression in the injection site as determined by luminance measurement. rAAVRec3 also had the greatest transduction volume, followed by AAV9 and rAAVRec4. The rAAVRec3 vector exhibits improved features over the currently popular natural variants and may have high potential for gene therapy for neurological disorders.

### Material and Methods

### AAV Vectors

Three primate-derived AAV variants, cy5 (cynomolgus macaque—variant 5), rh20 (rhesus macaque—variant 20) and rh39 were originally obtained from Dr. Guang-Ping Gao and the Gene Therapy Program Vector Core, Department of Medicine, University of Pennsylvania. These variants were selected due to their superior transduction efficiency (Lawlor et al., 2009). For the generation of hybrid recombinant capsids, fragments of capsid sequences that matched in all three vectors and AAV8 were shuffled around by using known restriction sites as described in (Charbel Issa et al., 2013). To generate hybrid AAV vectors, GFP was cloned into an AAV expression plasmid under the control of the CAG (hybrid CMV-chicken β-actin) promoter and containing woodchuck hepatitis virus posttranscriptional regulatory element (WPRE) and bovine growth hormone polyadenylation signal flanked by AAV-inverted terminal repeats. Human embryonic kidney 293 cells were co-transfected with three plasmids—AAV plasmid, appropriate helper plasmid encoding rep and cap (Rec1-4) genes or AAV1, AAV8, AAV9, and adenoviral helper pF Δ6—using standard CaPO4 transfection. rAAV vectors were purified from the cell lysate by ultracentrifugation through an iodixanol density gradient. Vectors were tittered using real-time PCR (ABI Prism 7700; Applied Biosystems, Foster City, Calif.) and diluted to 1.0×10¹³ vector genomes (vg)/mL for injection.

### AAV Titer Comparison

Each serotype virus was produced in five 150 mm plates. Virus genomic titer of each vector stock from each plate was determined by real-time PCR, and virus yield (virus genomic particles per cell, vg/cell) in each plate calculated.

### Mice

Fourteen week old male C57BL/6 mice (Charles River Laboratories, Wilmington, Mass., USA) were housed in groups of four under a 12 h light/dark cycle (lights off at 1800 hr), with food and water provided ad libitum. All use of animals was approved by the Ohio State University Animal Care and Use Committee, and was in accordance with the NIH guidelines.

rAAV Injection to Striatum

Mice were anaesthetized with a single dose of ketamine/xylazine (100 mg/kg and 20 mg/kg; i.p.) and placed on a Kopf stereotaxic frame. The injection coordinates for striatum were (from bregma): anterio-posterior, +1.0 mm; medio-lateral, ±1.7 mm; dorso-ventral, −3.5 mm (Franklin and Paxinos, 1997). 1 μL AAV vector (1×10¹³ vg/ml) was delivered bilaterally into both dorsal and ventral hippocampus at a rate of 0.1 μL/min using a 10 μL Hamilton syringe attached to Micro4 Micro Syringe Pump Controller (World Precision Instruments Inc., Sarasota, USA). Animals were monitored post-surgery until recovery from anaesthesia.

### Tissue Preparation for Immunohistochemistry

4 weeks after vector injection, mice were sacrificed by sodium pentobarbitone overdose (20 μL, i.p.) and perfused transcardially with 1×PBS followed by 4% PFA. Following cryoprotection in 30% sucrose, coronal brain sections of 40 μm were cut with a cryostat for immunohistochemistry.

### Immunohistochemistry

Brain sections were rinsed in 1×PBS containing 0.25% Triton X-100 (PBST) and blocked for 1 hour at room temperature in PBST containing 1% serum. After removal of the blocking buffer, the sections were incubated with rabbit anti-NeuN antibody (Abcam, 1:500) or goat anti-GFAP antibody (Santa Cruz Biotechnology, Inc., 1:100) overnight at 4° C. The next day, sections were washed thoroughly in PBST and incubated with the secondary antibodies, Cy3-conjugated goat anti-rabbit IgG (Jackson ImmunoResearch Laboratories, Inc., 1:250) or donkey anti-goat IgG-TR (Santa Cruz Biotechnology, Inc., 1:250) for 3 hours. Sections were then rinsed, mounted on slides, and cover slipped with fluorescent mounting medium (Vector Laboratories, Inc., Burlingame, Calif.).

### Confocal Microscopy

Brain sections were visualized on a confocal microscope (Olympus FluoView™ FV1000, Tokyo, Japan). The fluorescence of GFP, Cy3 and Texas Red® were sequentially excited using 488 nm argon laser and 543 nm HeNe laser. Images were collected sequentially using a ×40 oil immersion objective lens. Olympus Viewer was used to generate the merged images.

### Stereology

The transduction volume of brain tissue was quantified stereologically using the Cavalieri Estimator in Stereo Investigator 7 (MBF Bioscience, Willeston, Vt.). The area of each section containing GFP-positive immunoreactivity was outlined and markers were placed at a grid size of 100 μm to estimate the area of transduction within each section. The area in every 12th 40 μm section was measured (10-12 sections per brain measured, depending on transgene expression), then averaged and multiplied by the rostrocaudal distance between the first and last sections to give an estimate of transduction volume.

### Luminance of GFP Expression

The intensity of GFP expressed in each brain tissue was measured using the Collect Luminance Information command in Stereo Investigator 7 (MBF Bioscience, Willeston, Vt.). The image of the section with the most intense fluorescence for each brain was acquired and the GFP-expressing area was outlined. The luminance of each pixel inside the contour was measured and then averaged. Luminance has a range from 0 to 255 for each pixel. A black pixel has a luminance of 0, while a white pixel has a luminance of 255. For color pixels, the luminance is defined as (0.299*Red)+(0.579*Green)+(0.114*Blue).

### Statistical Analysis

Mean values from different experimental groups were compared using one-way ANOVA followed by pair-wise comparison by Student's t-test. All statistical analysis was done using the JMP software (SAS Institute Inc., Cary, N.C., USA), with significance set at P<0.05, All data are presented as means±standard error of the mean (S.E.M).

### Results

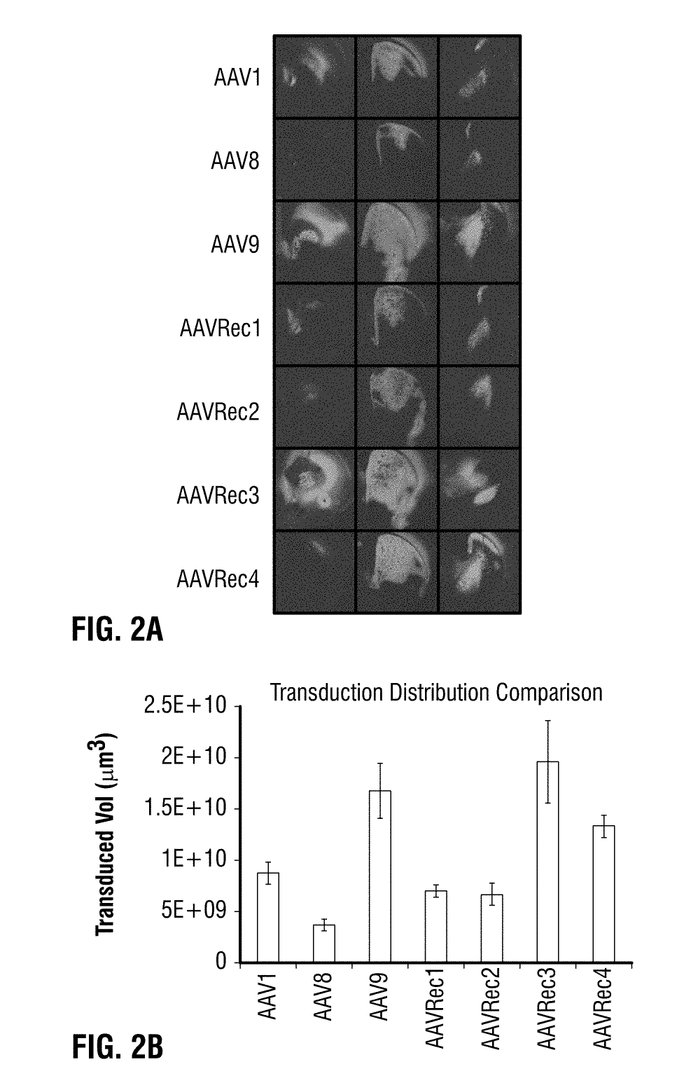

The transduction efficiency of four novel primate-derived hybrid recombinant AAV vectors (AAVRec1-4) was compared to vectors pseudotyped with wildtype capsids (AAV1, AAV8, AAV9) in the mouse brain. The volume of GFP-expressing tissue within the striatum was quantified using unbiased stereological methods. Overall, rAAVRec3 and AAV9 showed the most widespread GFP expression, followed by rAAVRec4. (FIG. 2; One-way ANOVA, P<0.0001). rAAVRec1 and rAAVRec2 produced comparable transduction volumes to AAV1, and AAV8 exhibited the least transduction volume. Intense GFP fluorescence was also observed in the globus pallidus, thalamus, cortex and thalamus of AAV9, rAAVRec3 and rAAVRec4 injected brains. A more detailed examination of rAAVRec3 injected brains revealed GFP positive fibers in the contralateral uninjected striatum, in the globus pallidus, and in the substantia nigra. In addition, GFP positive cells were observed in the thalamus and the cortex. Such cortical and thalamic cells transduction may occur through the retrograde transport of the vector through the corticostriatal and thalamostriatal afferents. Transduced cortical and thalamic neurons were detected as far as 1 mm away from the injection site, a distance that are considered by some to be too far to be explained by simple diffusion of the virus solution (Aschauer et al., 2013).

Interestingly, the novel serotype rAAVRec2, which has recently been shown to transduce both brown and white adipose tissues with the highest efficiency among the vectors tested (Liu et al., 2014), did not improve the transgene delivery targeting the brain. In contrast, rAAVRec3, rAAVRec4 and AAV9 transduce the brain with high efficiency but poorly transduced adipose tissues. The distinct tissue tropism of these engineered serotypes is a useful feature in expanding the current AAV vector toolkit for both basic research and clinical application.

In order to compare the intensity of transgene expression by various serotypes, the section with the most intense GFP fluorescence from each brain was selected, and the luminance was measured and averaged. rAAVRec3 showed the highest GFP fluorescence intensity, which is 2-fold higher than that mediated by AAV8 (FIG. 2A). rAAVRec4-mediated transgene expression was comparable to AAV9. The results indicate the maximal level of transgene protein expression achieved at the target site was higher using rAAVRec3 vectors. This could be due to increased transgene expression within transduced cells or a higher density of transduction (cells transduced per mm3) with the new hybrid recombinant serotype.

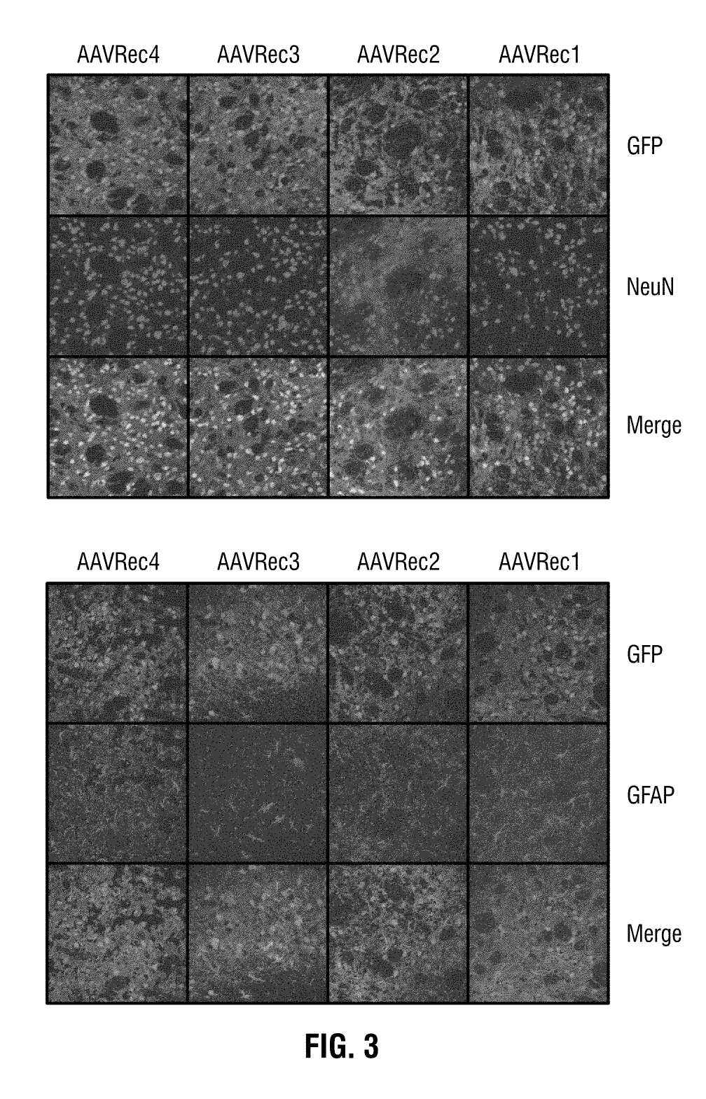

To determine the cellular tropism of rAAVRec1-4, confocal microscopy was used to visualize co-localization of GFP fluorescence and immunofluroscence of the different cell markers to different neural cell types using antibodies directed against cell-type-specific epitopes for neurons (NeuN) and astrocytes (GFAP). With all the serotypes tested, the majority of GFP-positive cells were immunoreactive with the neuronal marker NeuN with only 2-3 detectable astrocytic specific GFAP-positive cells per each section (FIG. 3), indicating that rAAVRec 1-4 predominantly transduce neurons. As expected, rAAVRec1-4 didn't alter the cellular tropism, which is consistent with the fact that the phenotype of transduced cells markedly depends on the promoter used (Lawlor et al., 2009). Transduction of astrocytes by AAV vectors might require the incorporation of glial-specific promoters. In addition, the brain region may also influence the cellular tropism of different AAV serotypes. For example, Aschauer and colleagues (2013) recently showed that while astrocytes in the cortex displayed higher GFP levels after transduction with AAV8 compared to AAV6 vectors, this difference in astrocytic transduction was not observed in the hippocampus (Aschauer et al., 2013). Interestingly, the same study showed that AAV8 was able to transduce astrocytes and oligodendrocytes and AAV1 showed some transduction of microglia. This could be a reflection of the different method employed. Whereas we report a more qualitative description of the transduction pattern (FIG. 3), Aschauer et al. assessed quantitatively the GFP signal intensity within each transduced cells of different cell types, thus even a small number of cells with high signal intensity may lead to a high cell-type specific expression. Nonetheless, the results clearly demonstrate the neurotropic nature of the four rAAVRec vectors, with rAAVRec3 also demonstrating moderate tropism for astrocytes (FIG. 3). During the production of these hybrid vectors, it was noticed that the different vectors lead to different production yield despite identical production methods by the same person (overall difference analyzed by ANOVA, P<0.0001). The results are presented in Table 1.

TABLE 1
Vector titers

| | | Significantly different groups |
|---|---|---|
| | Titer (×10⁵ | (P < 0.05) as determined by |
| Serotype | viral genome/cell) | Student's t-test |
| AAV1 | 0.38 ± 0.10 | AAVRec1, AAVRec2, AAVRec3 |
| AAV8 | 0.63 ± 0.14 | AAVRec1, AAVRec2, |
| AAV9 | 0.15 ± 0.05 | AAVRec1, AAVRec2, AAVRec3 |
| AAVRec1 | 1.32 ± 0.21 | AAVRec4, AAV1, AAV8, AAV9 |
| AAVRec2 | 1.70 ± 0.33 | AAVRec4, AAV1, AAV8, AAV9 |
| AAVRec3 | 1.25 ± 0.18 | AAVRec4, AAV1, AAV9 |
| AAVRec4 | 0.63 ± 0.13 | AAVRec1, AAVRec2, AAVRec3 |

Specifically, rAAVRec2 and rAAVRec1 exhibited the greatest yield compared to the other vectors. Although rAAVRec3 titer was almost 2-fold higher than AAV8, the difference did not reach statistical significance. Notably, although AAV9 produced highly efficient transduction in the brain, the titer produced was more than 8-fold lower than rAAVRec3 (P<0.001). The increased yield has practical relevance as it translates to greater transduction volume for the same production cost.

The present rAAV vectors generated by interchanging viral capsid protein sequences between different AAV serotypes may provide enhanced transduction efficiency and better production yield. The present hybrid vectors may be of use in circumventing immune responses as a second vector for re-administration. These hybrid vectors further expand the current AAV toolkit and are useful biological tools for neurological research.

It should be understood that the examples and embodiments provided herein are exemplary examples embodiments. Those skilled in the art will envision various modifications of the examples and embodiments that are consistent with the scope of the disclosure herein. Such modifications are intended to be encompassed by the claims.

All patents, patent applications and references cited throughout the specification are expressly incorporated by reference.

### REFERENCES

1. Srivastava A, Lusby E W, Berns M. 1983. Nucleotide sequence and organization of the adeno-associated virus 2 genome. J Virol 45:555-564.
2. Xiao X, Li J, McCown T J, Samulski R J. 1997. Gene transfer by adeno-associated virus vectors into the central nervous system. Exp Neurol 144:113-124.
3. Hermonat P L, Labow M A, Wright R, Berns M, Muzyczka N. 1984. Genetics of adeno-associated virus: isolation and preliminary characterization of adeno-associated virus type 2 mutants. J Virol 51:329-339.
4. Timpe J, Bevington J, Casper J, Dignam J D, Trempe J P. 2005. Mechanisms of adeno-associated virus genome encapsidation. Curr Gene Ther 5:273-284.
5. Bartlett J S, Wilcher R, Samulski R J. 2000. Infectious entry pathway of adeno-associated virus and adeno-associated virus vectors. J Virol 74:2777-2785.
6. Gao G, Vandenberghe L H, Wilson J M. 2005. New recombinant serotypes of AAV vectors. Curr Gene Ther 5:285-297.
7. Grimm D. 2002. Production methods for gene transfer vectors based on adeno-associated virus serotypes. Methods 28:146-157.
8. Rabinowitz J E, Rolling F, Li C, Conrath H, Xiao W, Xiao X, Samulski R J. 2002. Cross-packaging of a single adeno-associated virus (AAV) type 2 vector genome into multiple AAV serotypes enables transduction with broad specificity. J Virol 76:791-801.
9. Summerford C, Samulski R J. 1998. Membrane-associated heparan sulfate proteoglycan is a receptor for adeno-associated virus type 2 virions. J Virol 72:1438-1445.
10. Qing K, Mah C, Hansen J, Zhou S, Dwarki V, Srivastava A. 1999. Human fibroblast growth factor receptor 1 is a co-receptor for infection by adeno-associated virus 2. Nat Med 5:71-77.
11. Summerford C, Bartlett J S, Samulski R J. 1999. AlphaVbeta5 integrin: a co-receptor for adeno-associated virus type 2 infection. Nat Med 5:78-82.
12. Kaludov N, Handelman B, Chiorini J A. 2002. Scalable purification of adeno-associated virus type 2, 4, or 5 using ion-exchange chromatography. Hum Gene Ther 13:1235-1243.
13. Di Pasquale G, Davidson B L, Stein C S, Martins I, Scudiero D, Monks A, Chiorini J A. 2003. Identification of PDGFR as a receptor for AAV-5 transduction. Nat Med 9:1306-1312.
14. Samulski R J, Chang L S, Shenk T. 1989. Helper-free stocks of recombinant adeno-associated viruses: normal integration does not require viral gene expression. J Virol 63:3822-3828.
15. Carter B J. 2005. Adeno-associated virus vectors in clinical trials. Hum Gene Ther 16:541-550.
16. Fisher K J, Jooss K, Alston J, Yang Y, Haecker S E, High K, Pathak R, Raper S E, Wilson J M. 1997. Recombinant adeno-associated virus for muscle directed gene therapy. Nat Med 3:306-312.
17. Kaplitt M G, Leone P, Samulski R J, Xiao X, Pfaff D W, O'Malley K L, During M J. 1994. Long-term gene expression and phenotypic correction using adeno-associated virus vectors in the mammalian brain. Nat Genet 8:148-154.
18. Koeberl D D, Alexander I E, Halbert C L, Russell D W, Miller A D. 1997. Persistent expression of human clotting factor IX from mouse liver after intravenous injection of adeno-associated virus vectors. Proc Natl Acad Sci USA 94:1426-1431.
19. McCown T J, Xiao X, Li J, Breese G R, Samulski R J. 1996. Differential and persistent expression patterns of CNS gene transfer by an adeno-associated virus (AAV) vector. Brain Res 713:99-107.
20. Flannery J G, Zolotukhin S, Vaquero M I, LaVail M M, Muzyczka N, Hauswirth W W. 1997. Efficient photoreceptor-targeted gene expression in vivo by recombinant adeno-associated virus. Proc Natl Acad Sci USA 94:6916-6921.
21. Halbert C L, Rutledge E A, Allen J M, Russell D W, Miller A D. 2000. Repeat transduction in the mouse lung by using adeno-associated virus vectors with different serotypes. J Virol 74:1524-1532.
22. Senut M C, Suhr S T, Kaspar B, Gage F H. 2000. Intraneuronal aggregate formation and cell death after viral expression of expanded polyglutamine tracts in the adult rat brain. J Neurosci 20:219-229.
23. Smith-Arica J R, Thomson A J, Ansell R, Chiorini J, Davidson B, McWhir J. 2003. Infection efficiency of human and mouse embryonic stem cells using adenoviral and adeno-associated viral vectors. Cloning Stem Cells 5:51-62

What is claimed is:

1. A nucleic acid molecule coding for:
(i) one or more of the rAAVRec2 VP1, VP2, or VP3 sequences set forth in FIG. 1A; and/or
(ii) one or more of the rAAVRec3 VP1, VP2, or VP3 sequences set forth in FIG. 1A.

2. The nucleic acid of claim 1 wherein said nucleic acid comprises a recombinant adeno-associated virus (AAV) vector.

3. The nucleic acid molecule of claim 1 expressed within a recombinant mammalian cell wherein said cell expresses;
(i) one or more of the rAAVRec2 VP1, VP2, or VP3 sequences set forth in FIG. 1A; and/or
(i) one or more of the rAAVRec3 VP1, VP2, or VP3 sequences set forth in FIG. 1A.

4. A recombinant rAAVRec capsid comprising;
(ii) one or more of the rAAVRec2 VP1, VP2, or VP3 sequences set forth in FIG. 1A; and/or
(iii) one or more of the rAAVRec3 VP1, VP2, or VP3 sequences set forth in FIG. 1A.

5. A method for delivering a heterologous polynucleotide sequence into a mammal or a cell of a mammal comprising administering an adeno-associated virus (AAV) vector, said vector comprising:
(i) one or more of the rAAVRec2 VP1, VP2, or VP3 sequences set forth in FIG. 1A; and/or
(ii) one or more of the rAAVRec3 VP1, VP2, or VP3 sequences set forth in FIG. 1A; and
(iii) a heterologous polynucleotide sequence, to said mammal or a cell of said mammal, thereby delivering the heterologous polynucleotide sequence into the mammal or cell of the mammal.

6. The method of claim 5, wherein the mammalian cell is a neuronal cell.

7. The method of claim 5, wherein the mammalian cell is an adipocyte.

8. The method of claim 5, wherein the heterologous polynucleotide sequence is the wild type TSC1, wild type TSC2 or wild type SMA gene.

9. The method of claim 5 wherein the mammal is deficient in protein expression or function and in need of treatment.

10. The nucleic acid of claim 2 further comprising a pharmaceutically acceptable excipient, diluent and/or carrier.

11. A kit comprising the nucleic acid of claim 2.
